(19) United States
(12) Patent Application Publication
(10) Pub. No.: US 2022/0262524 A1
(43) Pub. Date: Aug. 18, 2022

(54) PARAMETER-ESTIMATION OF PREDICTOR MODEL USING PARALLEL PROCESSING

(71) Applicant: Innovative Emergency Management, Inc., Morrisville, NC (US)

(72) Inventors: Bradley T. Suchoski, Bel Air, MD (US); Steven A. Stage, Baton Rouge, LA (US); Heidi L. Gurung, Bel Air, MD (US); Prasith Baccam, Bel Air, MD (US)

(21) Appl. No.: 17/171,574

(22) Filed: Feb. 9, 2021

(51) Int. Cl. G16H 50/80; G06N 7/00

(52) U.S. Cl. CPC G16H 50/80; G06F 1/20; G06N 7/005

(57) ABSTRACT

Methods, systems, and computer programs are presented for accelerating large-scale analysis for predictive systems using program optimization and parallelization. Through GPU-optimization and processing parallelization, Markov Chain Monte Carlo (MCMC) methods are used to solve large, complex problems quickly. Faster run times are obtained for a compartmental epidemiological model applied to COVID-19 as compared to existing multi-threaded central processing unit (CPU) implementations. Due to the optimization and parallelization of the likelihood function in a Multiple-Try Metropolis (MTM) MCMC algorithm, a large quantity of simulations is executed quickly to estimate the parameter values in a Susceptible-Exposed-Infectious-Removed (SEIR) model with increased accuracy over existing solutions.

### TECHNICAL FIELD

The subject matter disclosed herein generally relates to methods, systems, and machine-readable storage media for forecasting the evolution of complex systems.

### BACKGROUND

Markov Chain Monte Carlo (MCMC) methods have emerged as one of the approaches for analyzing complex systems and performing predictions on the future evolution of such systems. MCMC estimates posterior distributions for use in Bayesian computations, which are practical applications useful in a number of fields, including machine learning, physics, and bioinformatics. Unfortunately, MCMC methods often suffer from slow run times when the data become large or the parameter values are found in complex distributions. This speed issue has prevented MCMC analysis from being used to solve interesting problems where MCMC methods would be valuable.

### BRIEF DESCRIPTION OF THE DRAWINGS

Various of the appended drawings merely illustrate example embodiments of the present disclosure and cannot be considered as limiting its scope.

FIGS. 1A-1B illustrate a Susceptible-Exposed-Infectious-Removed (SEIR) model for estimating the number of infected and dead individuals caused by the spread of a virus.

FIG. 2 illustrates the Metropolis Hastings algorithm for obtaining random samples from a probability distribution.

FIGS. 3A-3B illustrate a method for performing a large-scale Markov Chain Monte Carlo (MCMC) analysis using parallel processing of tasks in a graphics processing unit (GPU), according to some example embodiments.

FIG. 4 illustrates a sample system for performing embodiments.

FIG. 5 is a user interface for presenting predictions, according to some example embodiments.

FIG. 6 illustrates the accuracy of some example predictions.

FIG. 7 is a flowchart of a method for accelerating a large-scale MCMC estimation using parallel processing, according to some example embodiments.

FIG. 8 is a block diagram illustrating an example of a machine upon or by which one or more example process embodiments described herein may be implemented or controlled.

### DETAILED DESCRIPTION

Example methods, systems, and computer programs are directed to accelerating large-scale analysis for predictive systems using program optimization and parallelization. In one example, Markov Chain Monte Carlo (MCMC) analysis is accelerated using parallel processing with graphics processing units (GPU) to estimate the parameters of a Susceptible-Exposed-Infectious-Recovered (SEIR) model that forecasts the spread of COVID-19 infections.

Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will he evident to one skilled in the art, however, that the present subject matter may be practiced without these specific details.

Through GPU-optimization and processing parallelization, MCMC methods are used to solve large, complex problems quickly. Accelerated run times are obtained for a compartmental epidemiological model applied to COVID-19 as compared to existing multi-threaded central processing unit (CPU) implementations.

Through optimization via GPU processing and parallelization of the likelihood function in a Multiple-Try Metropolis (MTM) MCMC algorithm, a large quantity of simulations is executed to estimate the parameter values in the SEIR model with increased accuracy over existing solutions. Using the described techniques, the MCMC algorithms can be used in a large, real-world situation.

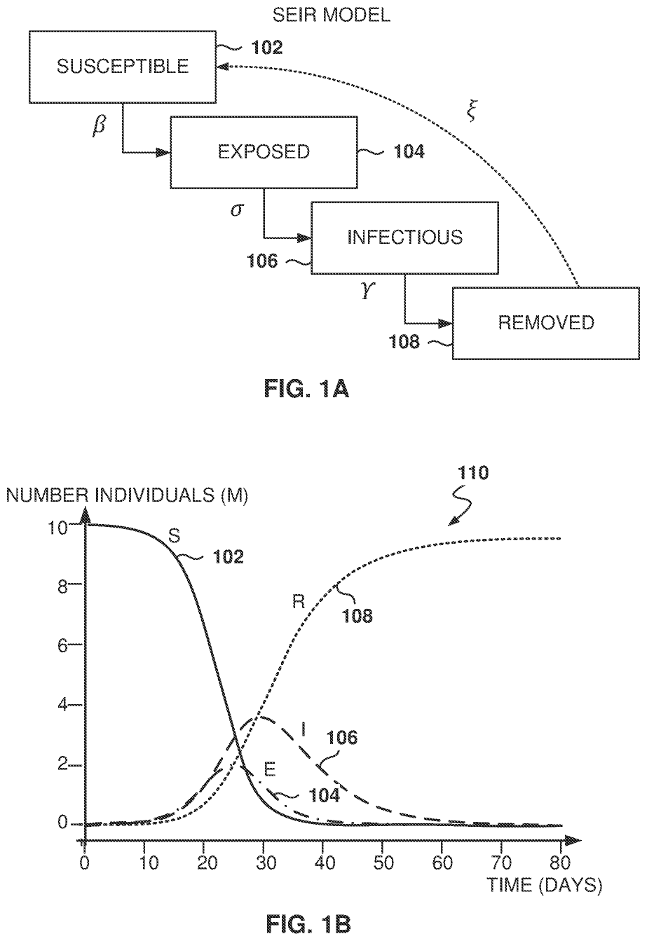

FIGS. 1A-1B illustrate a Susceptible-Exposed-Infectious-Recovered (SEIR) model for estimating the number of infected and dead individuals caused by the spread of a virus. The SEIR model has mainly four components: Susceptible (S) 102, Exposed (E) 104, Infectious (I) 106, and Removed (R) 108. S is the fraction of susceptible individuals (i.e., people able to contract the disease), E is the fraction of exposed individuals (i.e., those who have been infected but are not yet infectious), I is the fraction of infective individuals (i.e., those capable of transmitting the disease), and R is the fraction of recovered or removed individuals (i.e., those who have become immune or died).

Further, β is the infectious rate, which represents the probability of disease transmitting to a susceptible person from an infectious person; σ is the incubation rate, which represents the latent rate at which a person becomes infectious: γ is the recovery rate, which is determined by I/D (where D is the duration of infection); and ξ is the rate at which removed people become susceptible due to low immunity or other health related issues. In some example embodiments, ξ is set to zero but other values are also possible.

Below are the ordinary differential equations (ODES) associated with these four transition parameters.

$$\frac{dS}{dt} = - \frac{β SI}{N} + ξ R$$

$$\frac{dE}{dt} = - \frac{β SI}{N} - σ E$$

$$\frac{dI}{dt} = σ E - γ I$$

$$\frac{dR}{dt} = γ I - ξ R$$

Here, N is the total the population, calculated as S+E+I+R. Further, I/σ is the latent period of the disease T_{L}, and I/γ is the infectious period T_{I}. The value of R at the initial time, R_{0,} is calculated with the following equation:

$${R}_{0} = \frac{{β}_{0} σ}{( μ + σ ) ( μ + γ )}$$

FIG. 1B shows an example evolution of the pandemic. As shown, S 102 begins with the complete population and decays over time, R 108 begins at 0 and grows as people recover, E 104 grows first as more people are exposed and later declines as people recover, and I 106 grows as people get infected and later declines as people recover or die.

If the value of R 108 is greater than one, the evolution of I 106 will follow an approximate hell-shaped curve, with I 106 coming down at the end because the susceptible population is being exhausted, starting with S 102 at 100% of the population susceptible, and by the end, upwards of 65%, 75% of the population have been infected.

A number of SEIR tools and packages are available, such as the plot SIRModel package for plotting Markov chain SEIR models, the Biostats Python package for building SEIR models, and the Cornell multi-region SEIR model with mobility. Additionally, other tools solve the ODE equations.

In some example embodiments, a modeling tool referred to as BioSim was used to build the SEIR model with four main parameters: the reproductive number R(t) (that indirectly determines the force of infection variable β(t)), the latent period TL, and the infection start date where the entire population is assumed to be in the Susceptible S compartment except for a single person in exposed E. The BioSim application running the SEIR model is different from other SEIR models in that it is multithreaded in all instances (CPU or GPU) to run multiple cases with varying parameter sets simultaneously and is also capable of parallel processing using the GPU for further speed-up of calculations.

BioSim uses the concepts of compartments holding items, and the items may transition between compartments, similar to graphs of interconnected nodes with transitions between nodes that are counted at certain intervals. The SEIR model is simulated with BioSim defining the compartments for S, E, I, R and the transitions as illustrated in FIG. 1A.

In some solutions, the SEIR model does not take into consideration how long a person has been in one compartment. However, in the presented approach, the time spent in one compartment is considered to determine the probability of a transition, For example, the longer someone has been sick, the more probable is that the person will leave the I compartment.

R 108 can change dramatically over time because of multiple factors, e.g., behavior of the population, shutdowns, use of protective equipment, etc. The goal is to analyze the confirmed cases to calculate the value of R 108 at the current time.

In existing solutions, the SEIR models are executed once or twice to obtain the value of R and then use that value from that point on. But because of the changing value of R, these simple approaches become quickly inaccurate in their predictions. In some example embodiments, millions of simulations are performed to get the best R value, and the R value is updated frequently, such as daily. If the R value is calculated for each county in the United States (about 3200 counties), this means performing a large amount of simulations daily because the R value may change from county to county. With existing approaches, it becomes virtually impossible to keep accurate, current values of R for the whole country. For example, around 12 billion simulations would have to be performed daily to find the best values of R. Even doing fewer than all the counties each day would take a long number of simulations. For example, doing daily estimations for 426 regions (state or county), running 32 chains with 128 tries, each involving 100 startup iterations for 1 SEM simulation, plus 400 MCMC iterations requiring 2 SEW iterations, would mean a total number of 426*32*128*(100+2*400) SEM simulations, which is about 1.6 billion SEW simulations.

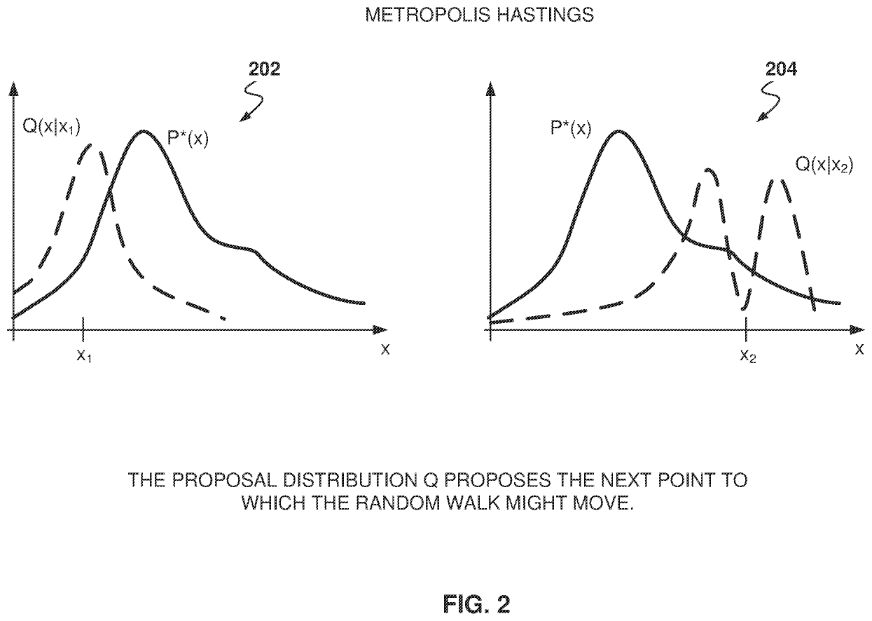

FIG. 2 illustrates the Metropolis Hastings algorithm for obtaining random samples from a probability distribution. A Markov process is a stochastic process for which predictions can be made regarding future outcomes based solely on its present state; such predictions are just as accurate as the ones that could be made knowing the process's full history. A Markov chain is a type of Markov process that has either a discrete state space or a discrete index set (often representing time). It is common to define a Markov chain as a Markov process in either discrete or continuous time with a countable state space.

A Markov Chain Monte Carlo (MCMC) method is an algorithm for sampling from a probability distribution. By constructing a Markov chain that has the desired distribution as its equilibrium distribution, it is possible to obtain a sample of the desired distribution by recording states from the chain. The more steps that are included, the more closely the distribution of the sample will match the actual desired distribution. Various algorithms exist for constructing chains, including the Metropolis -Hastings (MH) algorithm.

The changes of state of the system are called transitions, and the probabilities associated with state changes are called transition probabilities. A state space is a transition matrix describing the probabilities of particular transitions between states and includes an initial state (or initial distribution).

The MH algorithm is a Markov Chain Monte Carlo (MCMC) method for obtaining a sequence of random samples from a probability distribution P(x) from which direct sampling is difficult. This sequence can be used to approximate the distribution (e.g., to generate a histogram) or to compute an integral e.g., an expected value). MH and other MCMC algorithms are used for sampling from multi-dimensional distributions, especially when the number of dimensions is high.

The ME algorithm can draw samples from a probability distribution P(x), provided that a function “N, proportional to the density of P, is known and the values of” can be calculated. The ME algorithm works by generating a sequence of sample values in such a way that, as more and more sample values are produced, the distribution of values more closely approximates the desired distribution P(x). These sample values are produced iteratively, with the distribution of the next sample being dependent only on the current sample value (thus making the sequence of samples into a Markov chain). Specifically, at each iteration, the algorithm picks a candidate for the next sample value based on the current sample value. Then, with some probability, the candidate is either accepted (in which case the candidate value is used in the next iteration) or rejected (in which case the candidate value is discarded, and the current value is reused in the next iteration). The probability of acceptance is determined by comparing the values of the function f(x) of the current and candidate sample values with respect to the desired distribution P(x).

For illustration purposes a case where the proposal function is symmetric is described. Let f(x) be the likelihood function, which is proportional to the desired probability distribution P(x)(e.g., a target distribution).

At the initialization phase, an arbitrary point xₜ is chosen to be the first sample. Also chosen is an arbitrary probability density g(x/y (sometimes called Q(x/y)) that suggests a candidate for the next sample value x, given the previous sample value y. Further, g is assumed to be symmetric and satisfies g(x/y)=g(y/x). A usual choice is to let g(x/y) be a Gaussian distribution centered at y, so that points closer to y are more likely to be visited next, making the sequence of samples into a random walk. The function is referred to as the proposal density or jumping distribution.

After the initialization phase, an iterative loop is implemented, which includes the following:

Generate a candidate x' for the next sample by picking from the distribution g(x′/xt).

Calculate the acceptance ratio α=f(x')/f(xₜ), which will be used to decide whether to accept or reject the candidate. Because f is proportional to the density of P, we have that α=f(x')/f(xₜ)=P(x')/P(xₜ).

Accept or reject, which includes: generate a uniform random number u ∈[0, 1]; if u≤α, then accept the candidate by setting xₜ₊₁=X'; if u >α then reject the candidate and set xₜ₊₁=xₜ.

This algorithm proceeds by randomly attempting to move about the sample space, sometimes accepting the moves and sometimes remaining in place. The example in FIG. 2 illustrates how the proposal distribution Q proposes the next point to which the random walk might move.

The acceptance ratio a indicates how probable the new proposed sample is with respect to the current sample, according to the distribution P(x). If a move is attempted to a point that is more probable than the existing point (i.e., a point in a higher-density region of P(x)), the move will always be accepted. However, if a move to a less probable point is attempted, the move will sometimes be rejected, and the larger the relative drop in probability, the more likely it is to reject the new point.

MCMC methods are slow at analyzing large datasets and complex parameter sets. One way to overcome this problem is by using the Multiple-Try Metropolis (MTM) method, which is a modified form of the MH method designed to help the sampling trajectory converge faster, by increasing both the step size and the acceptance rate.

In the MTM algorithm, Q(x,y) is an arbitrary proposal function that satisfies that Q(x,y)>0 only if Q(y,x)>0. Additionally, π(x) is the likelihood function. The function w(x,y) is defined as w(x,y)=π(x)Q(x,y),λ(x,y) where λ(x,y) is a non-negative symmetric function in x and y that can be chosen by the user.

Given that the current state is x, the MTM algorithm includes the following operations:

1) Draw k independent trial proposals yₗ, . . . ,yₖ from Q(x.) and compute the weights w(yⱼ,x) for each of these proposals.

2) Select y from the yᵢ proposal with probability proportional to the weights.

3) Produce a reference set by drawing xᵢ, . . . , xₖ₋ₜ from the distribution Q(y,.), and set xₖ=x (the current point).

Accept y with probability r, where r is calculated as follows:

$$r = min ( 1 , \frac{w ( {y}_{1} , x ) + … + w ( {y}_{k} , x )}{w ( {x}_{1} , y ) + … + w ( {x}_{k} , y )} )$$

This method satisfies the detailed balance property and produces a reversible Markov chain with π(x) as the stationary distribution.

If Q(x,y) is symmetric (as is the case for the multivariate normal distribution), then choosing λ(x,y)=I/Q(x,y) gives w(x,y)=π(x).

One of the disadvantages of MTM is that it needs to compute the energy of 2k-I states at every step. If calculating the energy is slow, then this method will be slow.

In some example embodiments, the MTM variant of the MH algorithm is used while simulations are optimized to run on GRUs instead, or in combination with standard CPUs. The multiple-try aspect allows parallelization across CPUs, with a speed-up in getting simulation results.

The MTM-MCMC method used is based on a source-reconstruction plume dispersion model, which takes a time series of chemical concentration signals indicating where, when, and how much of a chemical agent was released and works backwards from those signals to find the most likely source of the chemical release.

The plume model is then run in a forward direction and recorded sensor data are compared to estimates from the MCMC analysis to identify the parameter sets most closely matching know, historical data.

The process of working backward to estimate SEIR parameter values and using those learned values to project forward to new case forecasts is based on the MCMC concepts of this plume dispersion model, with the multiple-try component added to further parallelize and increase the speed and accuracy of the model.

In some example embodiments, methods are presented for applying parallel solutions of compartment models to estimating parameters and forecasting new cases of the COVID-19 pandemic using recent confirmed case counts. In addition to the parallelization achieved through using a multiple-try algorithm to split the workload across numerous processes, both the BioSim software and the MCMC likelihood function were optimized to run on GPUs.

In 2013, Hall et al. proposed a Metropolis Monte Carlo (MMC) method for running molecular simulations using CUDA, a parallel computing platform and programming model developed by Nvidia. for general computing on GPUs. CUDA is used to run their CPU-GPU algorithm, with special manipulation of the GPU memory to decrease the use of system memory and swapping across devices. The system is designed with one GPU and one CPU running simultaneously, with the GPU parallelizing the MMC asynchronous to the CPU, which is responsible for running tasks that cannot be efficiently parallelized.

The CPU-GPU approach splits the work on the GPU into parallel threads. However, Hall's model is limited to the use of a single GPU, while the presented embodiments are scalable to any number of GPUs and has been run on single-, two-, four-, and eight-GPU systems.

In the presented implementations, as the CPU drives the analysis, there is no data dependency between the CPUs and the GPUs, and much of the original swapping between CPU and GPU referenced in this previous work is now conducted automatically within the drivers. A second difference between approaches is that Hall's method parallelized only the inner likelihood function, also referred to as the loss function, of the molecular simulations while each MCMC iteration is computed iteratively on the CPU, while in the presented embodiments, both the MCMC iteration loop and the likelihood function are parallelized, a key difference that results in faster execution and increased accuracy.

In some example embodiments, the MCMC analysis is implemented using simultaneous, individual, independent chains (e.g., 32 chains, but other values are also possible) with parameters randomly selected from the parameters' distributions. At each time step, a next step from a proposal distribution determined by the previous step location and covariance is selected. This results in more total work performed per MCMC iteration, but by using multiple independent chains, the proposal distribution more accurately estimates the local shape of the posterior distribution and reduces the total number of MCMC iterations needed for convergence.

Using MTM further reduces the number of iterations required for convergence. Both of these presented methods require more work, trading MCMC iterations for parallelism, but the additional work is parallelizable, making the simulations faster by running the iterations concurrently. In the presented model, the MCMC parameter selection and chain initialization takes place on the CPU, while the remaining calculations are run on the GPUs.

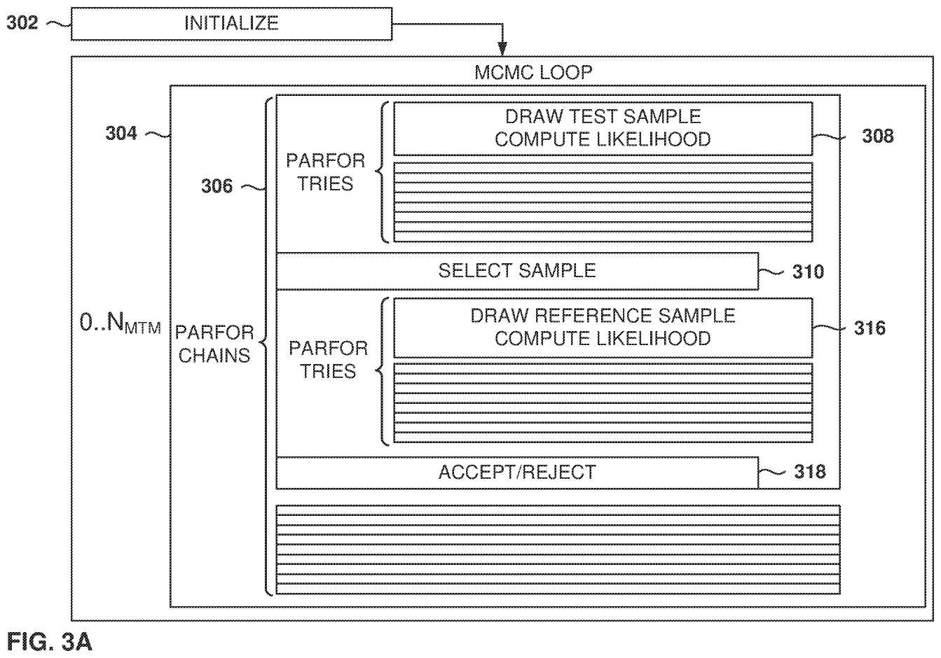

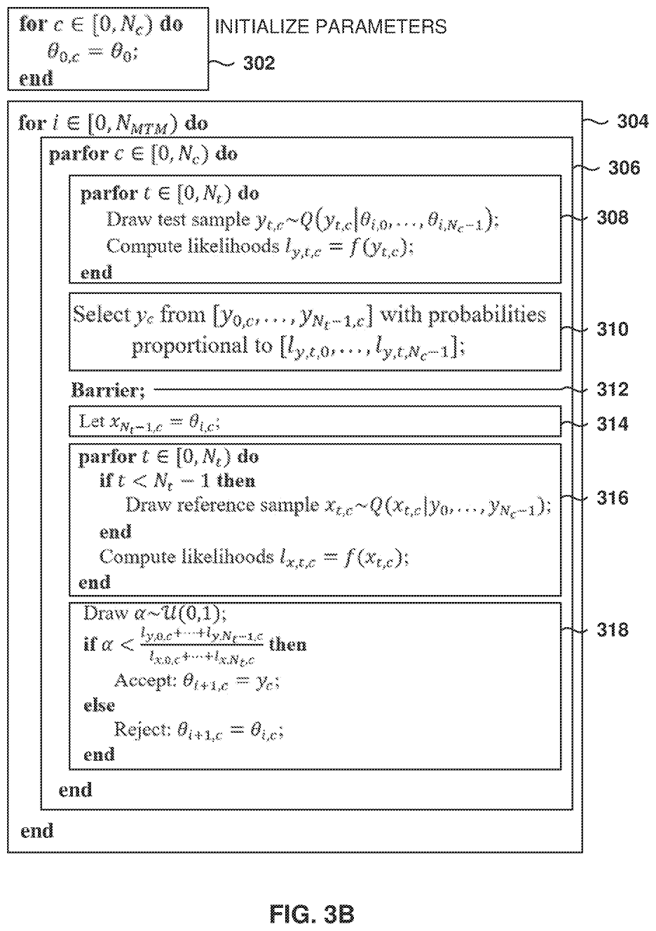

FIGS. 3A.-3B illustrate a method for performing a large-scale Markov Chain Monte Carlo (MCMC) analysis using parallel processing of tasks in a graphics processing unit (GPU), according to some example embodiments. Embodiments are presented with reference to COVID simulations, but the principles presented herein may be used for other applications that require estimating the behavior of complex systems.

The BioSim tool supports parallelized computations to run ensembles of simulations multithreaded on CPUs, or on GPUs for additional processing-speed improvements, allowing large numbers of simulations to be run quickly. Parallelizing is important for MCMC and other analyses that require large volumes of data. Further. BioSim is configured to support aged transitions, which means that individuals move between compartments stochastically and also based on their time in the compartment.

Further, the programming allows allocation of resources, and the resource allocations impact the outbreak. Resources can include vaccines, hospital beds, medical personnel, treatment availability, etc. This results in a non-Markov model that is very fast to compute.

In some embodiments, the implementation includes the following four components:

1. Development of an epidemiological model for predicting the evolution of the pandemic. The epidemiological model makes predictions based on the input parameter values, such as cumulative case counts over time.

2. Implementation of a parallelized MTM-MCMC method to estimate epidemiological model parameter values that best fit historic reported data.

3. An epidemiological model that tracks changes in epidemiological model parameters over time by using MTM-MCMC to fit parameters in a series of overlapping windows of historical data.

4. Application of these techniques to estimate the best model parameters that fit historic values and using the estimated model parameters to project future parameter values and case counts.

To find the most likely values of the epidemiological model parameters that explain the observed data, and to quantify the uncertainty in those values, a Markov Chain Monte Carlo (MCMC) method is used. MCMC provides several important benefits over other optimization algorithms, including being able to locate not only the optimal solution to some likelihood function, but also to estimate the shape of the entire posterior probability function π(θ|X) of some parameters θ given some observed data X. Further, the MCMC method does not rely on the ability to calculate or estimate any derivatives of the likelihood function, allowing for more complicated functions to be used. In some versions of MCMC, derivative information is used to accelerate convergence. However, the disadvantage of these versions is that the likelihood function is restricted to simple equations where the derivatives can be calculated. The algorithm presented can be used for any arbitrarily function and does not have this limitation. In some example embodiments, the likelihood function uses the solution to our SEW model. Further yet, the MTM variant of the standard MCMC method used provides a significant level of parallel computation well suited for execution on GPUs.

The MTM-MCMC method used here can be applied to other problems by changing the corresponding likelihood function. The likelihood function, also referred to simply as likelihood, measures the goodness of fit of a statistical model to a sample of data for given values of the unknown parameters. It is formed from the joint probability distribution of the sample but viewed and used as a function of the parameters only, thus treating the random variables as fixed at the observed values. The problem-specific likelihood function is both optimized and parallelized to run on one or more GPUs.

The MTM method is modified, and the reason for choosing the modified-MTM method over the MH method is that it trades parallelism fix likelihood function evaluations. The modified-MTM method typically has to perform more total likelihood function evaluations to achieve the same level of convergence as compared to the standard MH. However, the likelihood evaluations required at each MTM iteration can be computed in parallel, resulting in fewer MTM iterations required for convergence.

In addition to the parallelism provided by the likelihood function evaluations per outer iteration of this algorithm, the method also concurrently executes N_{c} independent chains to collect samples θ_{i,j} for i ∈[O,N_{MTM}] and j ∈[O,N_{c}]).

In some example embodiments, the MTM-MCMC method includes an initialization operation 302 to initialize parameters.

Further, an MCMC loop 304 is repeated for a plurality of iterations N_{MTM}. This MCMC loop 304 moves the current MCMC chain parameters θ around the posterior distribution space saving samples according to a likelihood acceptance criterion.

Within each MCMC loop 304, a plurality of chains 306 are examined in parallel. Each chain 306 includes parallel processing of a plurality of tries 308, followed by the selection of a sample 310, parallel processing of a plurality tries 316, followed by an accept or reject operation 318.

The tries 308 include drawing a test sample and then computing a first likelihood function. The tries 308 loop is the chain index loop (also referred to as a particle loop). Multiple chains are solved simultaneously (e.g., 32, but other values are also possible) and use the sample covariance of the current chain parameters to improve the jump proposal when the parameters have a high degree of correlation. This trades parallel work to reduce the number of outer MCMC iterations needed for convergence.

Further, the tries 316 include drawing a reference sample and then computing a second likelihood function. The tries 316 loop is for the MTM tries, where we “try” multiple proposal jumps per iteration instead of just one. This causes more parallel work per outer MCMC loop 304 but reduces the number of MCMC loops 304 needed for convergence. Instead of doing one likelihood value at a time, multiple likelihood values are calculated per iteration, but the likelihood. values are independent of one another, and the advantage is that they can be calculated in parallel.

FIG. 3B provides further details on the MTM-MCMC method. The data include the initial chain parameters θ_{0,c}, the likelihood function f(θ), and the proposal function Q(x|y). In some example embodiments, the chain parameter θ is the linear coefficient for calculating the R value (as discussed in more detail below), the outbreak start date, and if applicable to the configuration, the hospitalization rate and the death rate.

In some example embodiments, the MTM-MCMC method is expressed as follows:

```
for c ∈ [0,N_{c}) do
  θ_{0,c} = θ₀;
end
for i ∈ [0,N_{MTM}) do
  parfor c ∈ [0,N_{c}) do
    parfor t ∈ [0,Nₜ) do
      Draw test sample y_{t,c}~Q(y_{t,c}|θ_{i,0},...,θ_{i,N}_{_{c}}₋₁);
      Compute likelihoods l_{y,t,c} = f(y_{t,c});
    end
    Select y_{c} from [y_{0,c},...,y_{N}_{ₜ}_{-1,c}] with probabilities
    proportional to [l_{y,t,0},...,l_{y,t,N}_{_{c}}₋₁];
    Barrier;
    Let x_{N}_{ₜ}_{-1,c} = θ_{i,c};
    parfor t ∈ [0,Nₜ) do
      if t < Nₜ − 1 then
        Draw reference sample x_{t,c}~Q(x_{t,c}|y₀,...,y_{N}_{_{c}}₋₁);
      end
      Compute likelihoods l_{x,t,c} = f(x_{t,c});
    end
    Draw α~ [symbol]  (0,1);
    $if α < \frac{{l}_{y , 0 , c} \text{+} \text{…} \text{+} {l}_{y , {N}_{t} - 1 , c}}{{l}_{x , 0 , c} \text{+} \text{…} \text{+} {l}_{x , {N}_{t} , c}} then$
      Accept: θ_{i+1,c} = y_{c};
    else
      Reject: θ_{i+1,c} = θ_{i,c};
    end
  end
end
```

The initialization operation 302 includes a loop to initialize the chain parameters θ_{0,c}, e.g., with a predetermined value θ₀). The MCMC loop 304 is repeated for the plurality of iterations N_{MTM}, each iteration including the plurality of chains N_{c} 306 examined in parallel.

The tries 308 are calculated in parallel for a Nₜ number of tries. Within each try 308, a test sample y_{t,c} is selected (t is for the try within the tries 308 and c is for the chain within chain 306). The test sample y_{t,c} is drawn based on the N_{c} chain parameters θ_{t,c} as:

y_{t,c}˜Q(y_{t,c}|θ_{i,o}. . . ,θ_{i,N}_{_{c-1}})

Further, Q(θ′|θ) is the probability to try a move from a point ⁶ to a point θ′.

After the test sample y_{t,c} is drawn, the corresponding likelihood. l_{y,t,c}=f(y_{t,c}) is calculated. Here, f(θ) is the likelihood of the SEIR model. The SEER model is solved according to the parameters θ and then the method uses the mean square error of the predicted number of new cases compared to the reported number as the likelihood of the parameters θ.

Once the parallel calculations are completed for the tries 308. the test sample y_{c} is selected from all the calculated samples [y_{o,c}, . . . , y_{N}_{_{t-1,c}}]with probabilities proportional to the respected calculated likelihood functions [l_{y,t,o}, . . . , l_{y,t,N}_{_{c}}-1].

A barrier 312 is used for synchronization and separates the parallel computing for the tries 308 from the following parallel computations for the MTM tries 316. First, the reference sample x_{N}_{_{t-1,c}} is initialized at operation 314 with a value θ_{i,c}.

The MTM tries 308 include parallel computation for Nₜ tries, and within each try, a check is performed to determine if the try counter t is less than Nₜ₋ᵢ. If t is less than Nₜ₋ₗ then a reference sample x_{t,c} is drawn as:

x_{t,c}˜Q(x_{t,c}|yₒ. . . , y_{N}_{_{c-1}})

The MTM try 308 further includes computing the likelihoods l_{x,t,c}=f(x_{t,c}).

After the parallel computation of tries 316 are performed, at operation 318, the accept or reject decision is made, which includes generating the uniform random number u ∈[0,1] and performing the following check:

$$α < \frac{{l}_{y , 0 , c} + … + {l}_{y , {N}_{t} - 1 , c}}{{l}_{x , 0 , c} + … + {l}_{x , {N}_{t} , c}}$$

If the check is true, then the candidate is accepted, making θ_{i+1,c}=y_{c}. Otherwise, the candidate is rejected by setting θ_{i+1,c}=θ_{i,c}.

At the end, the result includes the parameter samples:

θ_{i,c}∀i ∈(O,N_{MTM},c ∈O, N_{c})

The predictive function is then determined based on the calculated θ_{i,c} values, and predictions are made using the determined predictive function.

It is noted that if Nₜ and N_{c} are equal to 1, then the MTM-MCMC method is the same as the traditional Metropolis Hastings MCMC algorithm, and if Nₜ is greater than 1 and N_{c} is equal to 1, then the MTM-MCMC method is equal to the MTM method. Therefore, the current embodiments where Nₜ and N_{c} are greater than 1 are improvements over the existing methods. Increasing Nₜ or N_{c} increases the amount of computational work required per outer MCMC loop. However, increasing either value results in faster convergence, thus requiring fewer total N_{MTM-}iterations.

Calculation of the likelihood for the SEIR model is how the MTM-MCMC method described above is applied to the SEIR model. Both the SEIR model and the MTM-MCMC analysis can be generalized to any problem complexity or domain; however, the likelihood function used by the MTM-MCMC analysis is specific to calculating historical case counts via the R(t) value of the SEIR model and projecting new cases.

The MTM-MCMC analysis is trying to capture the arbitrarily time-varying R(t) value, which is then used in the SEIR model to match the historical daily case counts (provided by Johns Hopkins University). The disease parameters are fit using windows where the process has been designed to ensure a symmetric proposal function across windows and simulation restarts.

It is important to maintain symmetry in the proposal function to ensure the assumptions of MTM are valid. By using small windows, we can have time-varying R(t) values to capture the change in the R(t) value within the pandemic over time. The likelihood function calculates the likelihood of each chain's current parameter set values and runs some number of chains to identify the most likely parameter set values.

While the projections are theoretically separate from the historical estimations, for computational simplicity both the historical and projected values are calculated using the final estimated R(t) value distributions. The historical case values are dependent on the time-varying value of R(t) within the window of interest. Projected case values are dependent on the final estimated R(t) value, The accuracy of projections is dependent on how the final R(t) value estimate is handled. In some example embodiments, a constant R(t) value is used, which means that the value of R(t) calculated for the given date (e.g., today) will continue to the end of the epidemic.

Regarding the implementation for the SEIR model, one of the aspects of the outbreak being analyzed is to be able to capture the time-varying nature of the model parameters, that is, as the pandemic evolves, the parameters may change due to changes in lockdowns, vaccines, treatments, etc. In particular, we know that the effective reproductive number, Rₑ(t), (or equivalently the transmissibility β(t)=Rₑ(t)/γ) is not constant, but changes over the course of an outbreak due to some of the aforementioned factors. The goal is to capture this dynamic nature of the changing reproductive timber, without making too many assumptions that would restrict the exact shape of the change, instead allowing the data to dictate the changes.

In some example embodiments, the outbreak is modeled over time using a series of overlapping constant size windows Wᵢ spanning a time t between a beginning time t_{b,i} and ending time (e.g., t_{b,i}≤t≤t_{e,i}). Within each window, the reproductive number R_{e,i}(t) is modeled as changing linearly over time as R_{e,i}(t)=Aᵢt+Bᵢ. The MTM-MCMC method is used to find the best estimate values for Aᵢ and Bᵢ that match the reported data for window Wᵢ. By allowing Aᵢ and Bᵢ to change from one window to the next, it is possible to capture the time varying nature of the outbreak. In some example embodiments, the window size is 20 days, but other embodiments may utilize other values, such as in the range from 5 to 100 days or more.

One problem with this approach is that the likelihood function for window Wᵢ involves using the solution to our SEIR model during t-window t_{b,0}≤t≤t_{b,i}. The state of the SEIR model during that time period is dependent on the solution of it for times t_{b,0}≤t≤t_{b,i}. To calculate the likelihood, two approaches are possible. First, run each SEIR model instance from t_{b,0} up to using t_{e,i} using Aⱼ and Bⱼ∀ⱼ≤i. The second approach, which is the chosen one, is to use a method for initializing the SEIR model at t_{b,i} and solve for times t_{b,i}≤t≤t_{e.i}.

The key insight needed to accomplish this is to realize that the state of the SEIR model at time t_{b,i} does not take on a single well-defined value. It is instead itself a random variable which can be calculated from the MCMC analysis parameters θ. To perform this approach, four parameters are added to the original MCMC analysis parameters so that \hat{θ}=[θ, i,j,k,U]. We are going to use the set of samples Sₜ^{w-1} saved from the previous window W_{w-1} of the state of the SEIR model in the middle of that window S(t_{m,w-1}). Then, for the current window, the following initial condition is used:

S(t_{b,w}) =Sᵢ^{w-1}+(Sⱼ^{w-1}−Sₖ^{w-1})U

To maintain the validity of the MCMC analysis at this point, a symmetric proposal function \hat{Q}(\hat{Q}′|\hat{Q})=\hat{Q}(\hat{Q}|\hat{Q}′) is chosen. One choice is to select the following:

$$\hat{Q} ( {\hat{θ}}^{′} \vert \hat{θ} ) = [ \begin{matrix}Q ( {θ}^{′} \vert θ ) \\ \\ \\ \\ \end{matrix} ]$$

Several considerations are noted regarding the application of MTM-MCMC method to the SEIR Model for estimating the value of B) and projections for new cases. The parameter search space used by the MTM algorithm consists of a set of variables that describe the four main parameters of our SEIR model: the reproductive number R(t), the latent period, the infectious period, and the infection start date. The latent period, infectious period, and start date are directly passed to the MTM algorithm as variables of the parameter space.

The parameter search space is configurable depending on what type of underlying epidemiological model is used. The MCMC algorithm works for any arbitrary parameter set. In some example embodiments, the SEIR model used includes three parameters in the search space: two coefficients for a linearly changing R value vs. time and the start date. Other example embodiments may use different or additional parameters such as hospitalization rates. Intensive Care Unit (ICU) case rates, case fatality rates, and projection R value coefficients.

An example of the two-coefficient linearly changing R value can be described by the equation:

R(t)=A(t)t+B(t)

Here, A(t) describes the slope of the change in R at time t and B(t) refers to an offset. The MCMC algorithm examines actual case data and finds the best values for A(t) and B(t) to fit the case data. The MCMC algorithm provides sample values of A(t) and B(t), and those values are converted into samples of R values that fit the data. We then use a method like Kernel Density Estimation (KDE) to approximate the continuous probability density function (PDF) from those R value samples. If the actual case data exhibit a growth in new cases, the MCMC finds that A(t) is positive and R increases relative to its previous value. Conversely, when the actual case data exhibit a decrease in new cases, A(t) is negative and R decreases relative to its previous value.

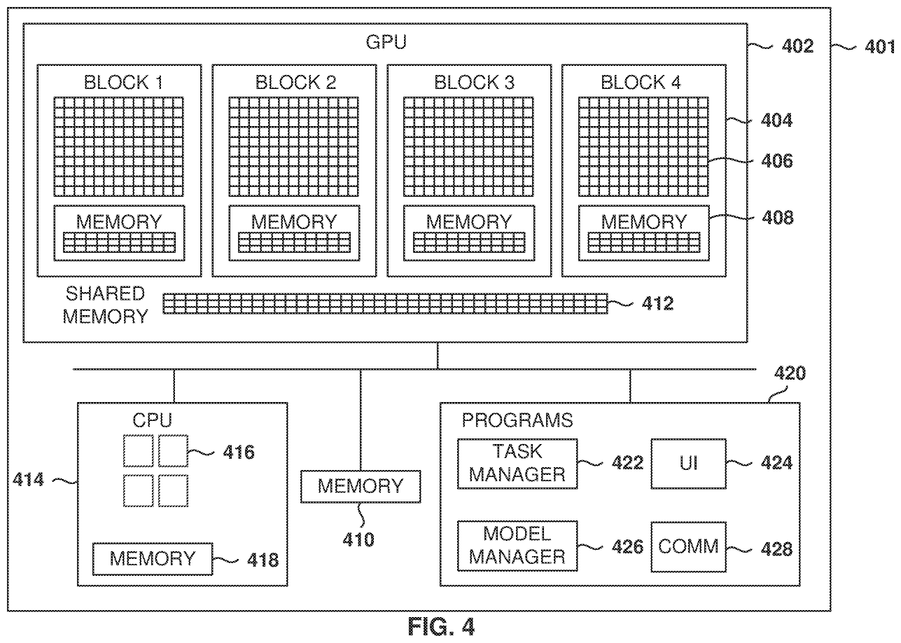

FIG. 4 illustrates a sample system for performing embodiments. Embodiments provide for the use of multiple GPUs to speed up the MTM-MCMC method, In the exemplary embodiment illustrated in FIG. 4, a computing device 401 includes one GPU 402, but other embodiments may utilize from two to ten GPUs or more.

Each GPU 402 includes a plurality of blocks 404 and shared memory 412. Each block 404 includes a plurality of cores 406 and a block memory 408 shared by all the cores 406 in the block 404. The cores 406 are independent from each other and can operate in parallel.

The CPU memory 412 may be used by the CPU 604 for local storage. in some example embodiments, the block memory 408 and the CPU memory 412 are used to store data for the calculations performed by the multiple threads during parallel processing.

The computing device 401 further includes CPU 416, memory 410, and a plurality of programs 420 (which may reside in memory 410 or in permanent storage). The CPU 416 includes one or more cores 416 and a CPU memory 418. The CPU 416 is used to execute programs 420.

The programs 420 include a task manager 422, a user interface 424, a model manager 426, and a communication manager 428. The task manager 422 manages the activities of the CPUs for evaluating one or more models and assigns tasks to the CPU 402 for parallel processing. The task manager 422 coordinates the loading of the parallel tasks into the CPUs. For example, the task manager 422 assigns threads for operations of the tries 308 or the tries 316.

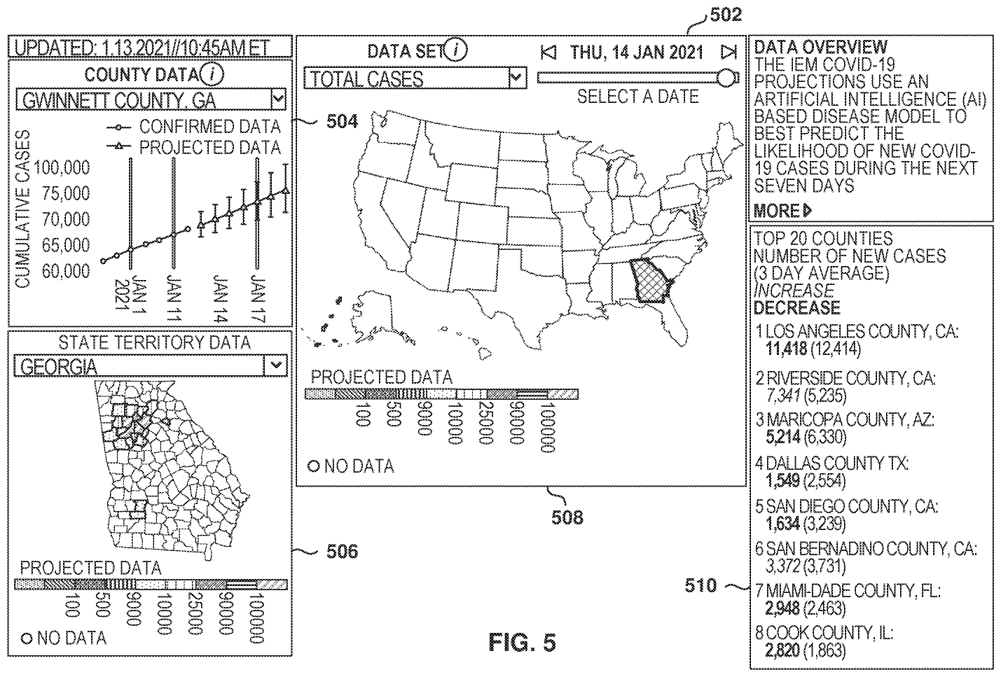

The user interface (UT) 424 may be used to configure operations for setting up the MTM-MCMC method and for presenting results. in one example embodiment, a UI 502 provides a user interface, as described below with reference to FIG. 5. to follow the evolution of the pandemic and present estimates for the evolution of the pandemic.

The model manager 426 manages the model parameters for performing the MTM-MCMC method. The communications manager 428 manages the communication operations within the computing device 401 as well as communications via a network (not shown).

The computing device 401 performs the operations of the MTM-MCMC method. By utilizing a large number of cores, the number of model parameters evaluated in parallel is greatly increased.

In some example embodiments, the threads may utilize the data from other threads, e.g., by sharing data in the block memory 408 or the GPU shared memory 412. For example, one thread may analyze the state of another thread.

In some example experiments, eight GPU's 402 were utilized, each GPU having 4400 cores, and between 10,000 and 100,000 threads were utilized for the parameter evaluation.

In one example embodiment, one system utilized one Intel Core i9-10920X CPU with 12 physical cores and hyperthreading, 128 GB of main memory, and four Nvidia RTX 2080 Ti GPUs. In another configuration, an EC2 instance was used on AWS (p3.16× large), with two Intel Xeon E5-2686 v4 CPU's with 16 physical cores each and hyperthreading, 500 GB of main memory, and eight Nvidia Tesla V100-SXM2-16GB GPUs.

FIG. 5 is a user interface 502 for presenting predictions, according to some example embodiments. In some example embodiments, the user interface 502 provides information by county, but other embodiments may provide information also by state or for the nation as a whole.

The user interface 502 includes a window 504 for presenting county data, a window 506 for presenting state information, a window 508 for presenting nationwide information, and a window 510 for presenting ranking information, e.g. by county.

The window 504 includes a drop-down menu for selecting the county and presenting a chart with the evolution of the pandemic, with time on the horizontal axis and the number of cases on the vertical axis. The chart further shows the evolution of the pandemic to date and the estimated evolution of the pandemic for future dates, within a range of possible values.

It is noted that the projections for a large number of regions may be updated daily, e.g., 375 counties, 50 states and 3 US territories but other number of jurisdictions may be calculated, an operation that is possible due to the improvements presented herein utilizing the MTM-MCMC method to use GPUs for fast parameter evaluation.

The window 506 includes a drop-down menu for selecting the state and a map of the state where each county is color-coded based on the number of projected cases, which provides a quick visual representation for determining where the pandemic is evolving better or worse.

The window 508 includes a drop-down menu for selecting whether to present information by the total number of cases or by number of cases per capita, a time bar to select a date for the number of cases, and a map with color-coded information by county. Further, the window 510 includes a list of the top counties by number of new cases, and for each county, the number of cases is presented along with the three-day average. The number of cases is color-coded to show whether the number of cases is increasing or decreasing.

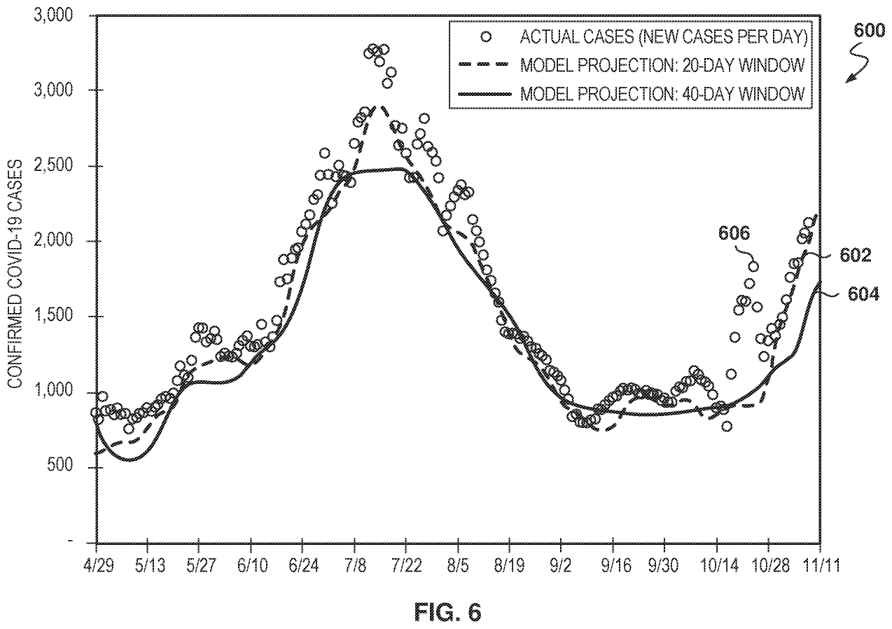

FIG. 6 illustrates the accuracy of some example predictions. In some experiments, the accuracy of the MTM-MCMC method to fit historical confirmed COVID-19 cases was evaluated by examining the difference between projected cases and actual cases. Because actual case counts can vary greatly daily, a seven-day average of daily new cases, centered around the date of interest, was calculated. FIG. 6 shows the seven-day average of daily new COVID-19 cases 606, and the model projections, using a 20-day window 602 or a 40-day window 604, for estimating R(t) over time.

Table 1 shows statistical metrics of fit for both the 20-day and the 40-day windows using the difference between projected and actual cases. Both the 40-day and 20-day windows under-predicted the model, as the middle 50% of results (area between the 25th and 75th percentiles) are both at least 6.2% lower than the actual historical amount. This can be rationalized that in the early stages of the pandemic the reporting process was prone to have large amounts of backlogged data all reported at once.

TABLE 1

| | | | | 25^{th} | 75^{th} |
|---|---|---|---|---|---|
| | Avg | Median | STD | pctl | pctl |
| Historical data (new | −9.7% | −7.9% | 9.4% | −13.1% | −4.1% |
| cases) 20-day window | | | | | |
| Historical data (new | −13% | −13.1% | 11.1% | −19.1% | −6.2% |
| cases) 40-day window | | | | | |
| Projections | −1.2% | −0.7% | 2.9% | −2.0% | 0.2% |
| (cumulative cases) | | | | | |

The cumulative projection modeling had greater overall accuracy as the middle 50% of results were between 2.0% lower and 0.2% higher than the actual cumulative cases at that date. That, combined with the mean and median being within the 25th and 50th percentile, shows that while on average this model slightly underpredicts the cumulative total, the variance is very small and is less prone to have large outliers of under- or overprediction. Because we are calculating a cumulative case total, better percentage differences are expected when comparing cumulative predictions versus new daily observations.

Further, a 10-day window was also examined for estimating RN over time, and while the 10-day window resulted in a slightly better fit than the 20-day window, its projections had too many rapid changes that would wildly affect future projections. Additionally, with a 10-day window, the overlap with the previous window would be just 5 days, which may cause too much variation in future projections.

The process of optimizing and parallelizing the computation of the likelihood function for the MTM-MCMC problem can be leveraged for many different types of problems, such as the plume source analysis problem and DNA analysis. Further, the MTM-MCMC method may be used with additional parametrization, such as using additional compartments, aged transitions, and resource constraints to more closely match real-world scenarios and support more accurate estimates of resource needs, such as hospital beds, ventilators, medication requirements, etc.

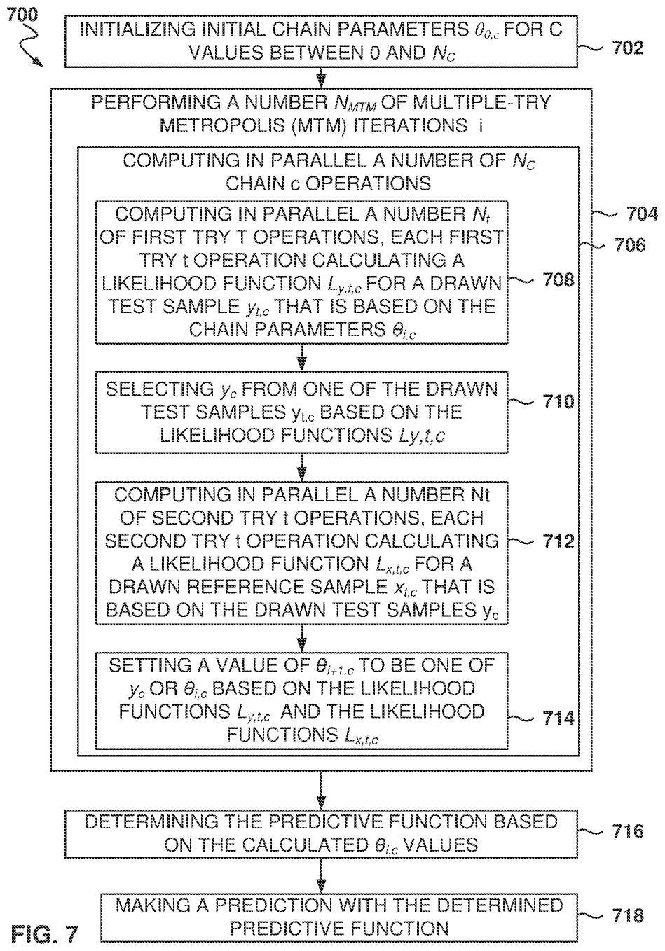

FIG. 7 is a flowchart of a method 700 for accelerating a large-scale MCMC estimation using parallel processing, according to some example embodiments. While the various operations in this flowchart are presented and described sequentially, one of ordinary skill will appreciate that some or all of the operations may be executed in a different order, be combined or omitted, or be executed in parallel.

Operation 702 is for initializing initial chain parameters θ_{0,c} for c values between O and N.

From operation 702, the method 700 flows to operation 704 for performing a number N_{MTM} of multiple-try metropolis (MTM) iterations i, each iteration i comprising operation 706 for computing in parallel a number of N_{c} chain c operations, each chain c operation comprising:

Operation 708 for computing in parallel a number Art of first try t operations, each first try t operation calculating a likelihood function l_{y,t,c} for a drawn test sample yₜ, that is based on the chain parameters θ_{i,c};

Operation 710 for selecting y_{c} from one of the drawn test samples y_{t,c} based on the likelihood functions l_{y,t,c};

Operation 712 for computing in parallel a number Nₜ of second try t operations, each second try t operation calculating a likelihood function l_{x,t,c} for a drawn reference sample x_{t,c} that is based on the drawn test samples y_{c}; and

Operation 714 for setting a value of θ_{i+l,c} to be one of y_{c} or θ_{i,c} based on the likelihood functions l_{y,t,c} and the likelihood functions l_{x,t,c}.

From operation 704, the method 700 flows to operation 716 to determine the predictive function based on the calculated θ_{i,c} values.

From operation 716, the method 700 flows to operation 718 for making a prediction with the determined predictive function.

In one example, the predictive function is for estimating the future number of cases during a pandemic.

In one example, the predictive function is for a reproductive number R(t) during the pandemic, wherein R(t) is a linear function with respect to t.

In one example, a window of time is predefined for drawing samples of number of cases in the pandemic.

In one example, each first try t operation comprises: drawing a test sample from a proposal function Q(y_{t,c}|θ_{i,o}, θ_{i,N}_{_{c}}-1); and calculating the likelihood function l_{y,t,c} for the drawn test sample y_{t,c}.

In one example, selecting y_{c} from one of the drawn test samples y_{t,c} comprises: selecting the y_{c} based on respective probabilities of likelihood functions [l_{y,t,o}, . . . ,l_{y,t,N}_{_{c-1}}].

In one example, the method 700 further comprises, before the second try t operations, making x_{Nt-1,c} equal to θ_{i,c}.

In one example, the second try t operation comprises: when t is less than Nᵢ-1 then drawing the reference sample x_{t,c} that is from a proposal function Q(x_{t,c}|y₀, . . . , y_{N}_{_{c}}).

Another general aspect is for a system that includes a memory comprising instructions and one or more computer processors. The instructions, when executed by the one or more computer processors, cause the one or more computer processors to perform operations comprising: initializing initial chain parameters θ_{0,c} for c values between 0 and N_{c}; performing a number N_{MTM} of multiple-try metropolis (MTM) iterations i, each iteration i comprising: computing in parallel a number of N_{c} chain c operations, each chain c operation comprising: computing in parallel a number Nₜ of first try t operations, each first try t operation calculating a likelihood function l_{y,t,c} for a drawn test sample y_{t,c} that is based on the chain parameters θ_{i,c}; selecting y_{c} from one of the drawn test samples y_{t,c} based on the likelihood functions l_{y,t,c}; computing in parallel a number Nₜ of second try t operations, each second try t operation calculating a likelihood function l_{x,t,c} for drawn reference sample x_{t,c} that is based on the drawn test samples y_{t,c}; and setting a value of θ_{i+1,c} to be one of y_{c} or θ_{t,c} based on the likelihood functions l_{y,t,c} and the likelihood functions l_{x,t,c}; determining a predictive function based on the calculated θ_{i,c} values; and making a prediction with the determined predictive function.

In yet another general aspect, a machine-readable storage medium (e.g., anon-transitory storage medium) includes instructions that, when executed by a machine, cause the machine to perform operations comprising: initializing initial chain parameters θ_{0,c} for c values between O and N_{c}; performing a number N_{MTM} of multiple-try metropolis (MTM) iterations i, each iteration i comprising: computing in parallel a number of N_{c} chain c operations, each chain c operation comprising: computing in parallel a number Nₜ of first try t operations, each first try t operation calculating a likelihood function l_{y,t,c} for a drawn test sample y_{t,c} that is based on the chain parameters θ_{i,c}; selecting y_{c} from one of the drawn test samples based on the likelihood functions l_{y,t,c}; computing in parallel a number Nₜ of second try t operations, each second try t operation calculating a likelihood function l_{x,t,c} for a drawn reference sample x_{t,c} that is based on the drawn test samples y_{c}; and setting a value of θ_{i+1,c} to be one of y_{c} or θ_{i,c} based on the likelihood functions l_{y,t,c} and the likelihood functions l_{x,t,c}; determining a predictive function based on the calculated θ_{i,c} values; and making a prediction with the determined predictive function.

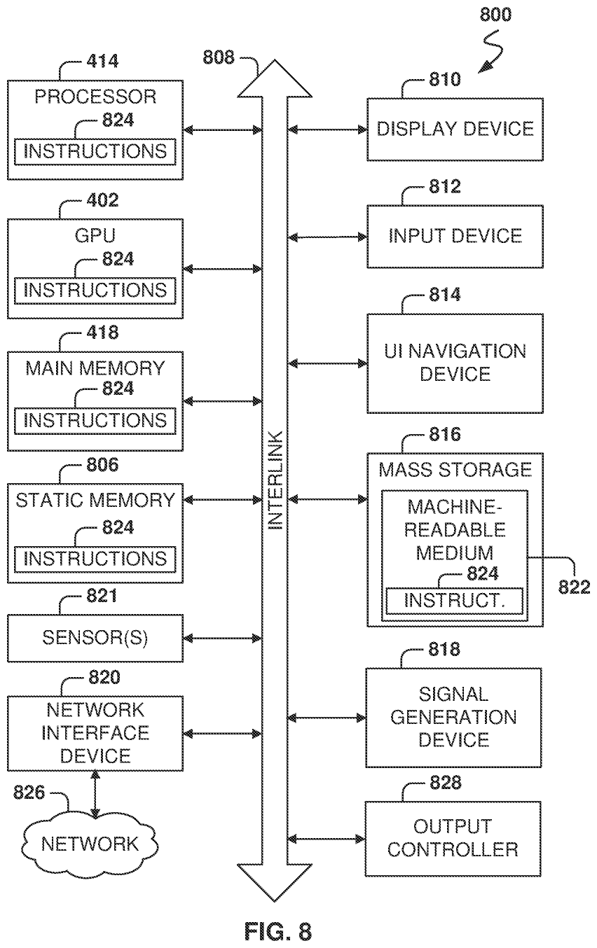

FIG. 8 is a block diagram illustrating an example of a machine 800 upon or by which one or more example process embodiments described herein may be implemented or controlled. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in a peer-to-peer (P2P) (or other distributed) network environment. Further, while only a single machine 800 is illustrated, the term “machine” shall also be taken to include any collection of machines that individually or jointly execute a set (or multiple sets) of instructions to perform any one or more of the methodololgies discussed herein, such as via cloud computing, software as a service (SaaS), or other computer cluster configurations.

Examples, as described herein, may include, or may operate by, logic, a number of components, or mechanisms. Circuitry is a collection of circuits implemented in tangible entities that include hardware (e.g., simple circuits, gates, logic). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits) including a computer-readable medium physically modified (e.g., magnetically, electrically, by moveable placement of invariant massed particles) to encode instructions of the specific operation. In connecting the physical components, the underlying electrical properties of a hardware constituent are changed (for example, from an insulator to a conductor or vice versa). The instructions enable embedded hardware (e.g., the execution units or a loading mechanism) to create members of the circuitry in hardware via the variable connections to carry out portions of the specific operation when in operation. Accordingly, the computer-readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry, at a different time.

The machine (e.g., computer system) 800 may include a hardware processor 414 (e.g., a central processing unit (CPU), a hardware processor core, or any combination thereof), a graphics processing unit (GPU) 402, a main memory 418, and a static memory 806, some or all of which may communicate with each other via an interlink (e.g., bus) 808. The machine 800 may further include a display device 810, an alphanumeric input device 812 (e.g., a keyboard), and a user interface (UI) navigation device 814 (e.g., a mouse). In an example, the display device 810, alphanumeric input device 812, and UI navigation device 814 may be a touch screen display. The machine 800 may additionally include a mass storage device (e.g., drive unit) 816, a signal generation device 818 (e.g., a speaker), a network interface device 820, and one or more sensors 821, such as a Global Positioning System (GPS) sensor, compass, accelerometer, or another sensor. The machine 800 may include an output controller 828, such as a serial (e.g., universal serial bus (USB)), parallel, or other wired or wireless (e.g., infrared (IR), near field communication (NFC)) connection to communicate with or control one or more peripheral devices (e.g., a printer, card reader).

The mass storage device 816 may include a machine-readable medium 822 on which is stored one or more sets of data structures or instructions 824 (e.g., software) embodying or utilized by any one or more of the techniques or functions described herein, The instructions 824 may also reside, completely or at least partially, within the main memory 418, within the static memory 806, within the hardware processor 414, or within the GPU 402 during execution thereof by the machine 800. In an example, one or any combination of the hardware processor 414, the GPU 402, the main memory 418, the static memory 806, or the mass storage device 816 may constitute machine-readable media.

While the machine-readable medium 822 is illustrated as a single medium, the term “machine-readable medium” may include a single medium, or multiple media (e.g., a centralized or distributed database, and/or associated caches and servers) configured to store the one or more instructions 824.

The term “machine-readable medium” may include any medium that is capable of storing, encoding, or carrying instructions 824 for execution by the machine 800 and that cause the machine 800 to perform any one or more of the techniques of the present disclosure, or that is capable of storing, encoding, or carrying data structures used by or associated with such instructions 824. Non-limiting machine-readable medium examples may include solid-state memories, and optical and magnetic media. In an example, a massed machine-readable medium comprises a machine-readable medium 822 with a plurality of particles having invariant (e.g., rest) mass. Accordingly, massed machine-readable media are not transitory propagating signals. Specific examples of massed machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may he provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

What is claimed is:

1. A computer-implemented method for calculating parameters of a predictive function, the method comprising:
initializing initial chain parameters θ_{0,c} for c values between 0 and N_{c};
performing a number A^{I}mml of multiple-try metropolis (MTM) iterations i, each iteration i comprising:
computing in parallel a number of N_{c} chain c operations, each chain c operation comprising:
computing in parallel a number Nₜ of first try t operations, each first try t operation calculating a likelihood function l_{y,t,c} for a drawn test sample y_{t,c} that is based on the chain parameters θ_{i,c};
selecting y_{c} from one of the drawn test samples y_{t,c} based on the likelihood functions l_{y,t,c};
computing in parallel a number Nₜ of second try t operations, each second try t operation calculating a likelihood function l_{x,t,c} for a drawn reference sample x_{t,c} that is based on the drawn test samples y_{c}; and
setting a value of θ_{i+t,c} to be one of y_{c} or θ_{i,c} based on the likelihood functions l_{y,t,c} and the likelihood functions l_{x,t,c};
determining the predictive function based on the calculated θ_{i,c} values; and
making a prediction with the determined predictive function.

2. The computer-implemented method as recited in claim 1, wherein the predictive function is for estimating future number of cases during a pandemic.

3. The computer-implemented method as recited in claim 2, wherein the predictive function is for a reproductive number R(t) during the pandemic, wherein R(t) is a linear function with respect to t.

4. The computer-implemented method as recited in claim 2, wherein a window of time is predefined for drawing samples of a number of cases in the pandemic.

5. The computer-implemented method as recited in claim I, wherein each first try t operation comprises:
drawing a test sample y_{t,c} from a proposal function Q(y_{t,c}|θ_{i,0,...,}θ_{i,N}_{_{c-1}}); and
calculating the likelihood function l_{y,t,c} for the drawn test sample y_{t,c}.

6. The computer-implemented method as recited in claim 1, wherein selecting y_{c} from one of the drawn test samples y_{t,c} comprises:
selecting the y_{c} based on respective probabilities of likelihood functions [l_{y,t,0,} . . . ,l_{y,t,N}_{_{c-1}}].

7. The computer-implemented method as recited in claim 1, further comprising:
before the second try t operations, making x_{Nt-t,c} equal to θ_{i,c}.

8. The method as recited in claim 1. wherein the second try t operation comprises:
when t is less than Nₜ₋₁ then drawing the reference sample x_{t,c} that is from a proposal function Q(x_{t,c}|y₀, . . . , y_{N}_{_{c-1}}).

9. A system comprising:
a memory comprising instructions, and
one or more computer processors, wherein the instructions, when executed by the one or more computer processors, cause the system to perform operations comprising:
initializing initial chain parameters θ_{0,c} for c values between 0 and N_{c};
performing a number N_{MTM} of multiple-try metropolis (MTM) iterations i, each iteration i comprising:
computing in parallel a number of N_{c} chain c operations, each chain c operation comprising:
computing in parallel a number Nₜ of first try I operations, each first try t operation calculating a likelihood function l_{y,t,c} for a drawn test sample y_{c} that is based on the chain parameters θ_{t,c};
selecting y from one of the drawn test samples y_{t,c} based on the likelihood functions l_{y,t,c};
computing in parallel a number Nₜ of second try t operations, each second try t operation calculating a likelihood function l_{x,t,c} for a drawn reference sample x_{t,c} that is based on the drawn test samples y_{c}; and
setting a value of θ_{i+l,c} to be one of y_{c} or θ_{i,c} based on the likelihood functions l_{y,t,c} and the likelihood functions l_{x,t,c};
determining a predictive function based on the calculated θ_{i,c} values; and
making a prediction with the determined predictive function.

10. The system as recited in claim 9, wherein the predictive function is for estimating a future number of cases during a pandemic.

11. The system as recited in claim 10, wherein the predictive function is for a reproductive number R(t) during the pandemic, wherein R(t) is a linear function with respect to t.

12. The system as recited in claim 10, wherein a window of time is predefined for drawing samples of a number of cases in the pandemic.

13. The system as recited in claim 9, wherein each first try r operation comprises:
drawing a test sample y_{t,c} from a. proposal function Q(y_{t,c}|θ_{i,0,} . . . ,θ_{i,N}_{_{c-1}}); and
calculating the likelihood function l_{y,t,c} for the drawn test sample y_{t,c}.

14. The system as recited in claim 9, wherein selecting y_{c} from one the drawn test samples y_{t,c} comprises:
selecting they. based on respective probabilities of likelihood functions [l_{y,t,0}, l_{y,t,N}_{_{c}}-1].

15. The system as recited in claim 9, wherein the instructions further cause the one or more computer processors to perform operations comprising:
before the second try t operations, making x_{Nt-1,c} equal to θ_{i,c}.

16. A tangible machine-readable storage medium including instructions that, when executed by a machine, cause the machine to perform operations comprising:
initializing initial chain parameters θ_{0,c} for c values between 0 and N_{c};
performing a number N_{MTM} of multiple-try metropolis (MTM) iterations each iteration i comprising:
computing in parallel a number of N_{c} chain c operations, each chain c operation comprising:
computing in parallel a number At of first try t operations, each first try t operation calculating a likelihood function l_{y,t,c} for a drawn test sample y_{t,c} that is based on the chain parameters θ_{i,c};
selecting y from one of the drawn test samples y_{t,c} based on the likelihood functions l_{y,t,c};
computing in parallel a number Nₜ of second try t operations, each second try t operation calculating a likelihood function l_{x,t,c} for a drawn reference sample x_{t,c} that is based on the drawn test samples y_{c}; and
setting a value of θ_{i+1,c} to be one of y_{c} or θ_{i,c} based on the likelihood functions l_{y,t,c} and the likelihood functions l_{x,t,c};
determining a predictive function based on the calculated θ_{i,c} values; and
making a prediction with the determined predictive function.

17. The tangible machine-readable storage medium as recited in claim 16, wherein the predictive function is for estimating a future number of cases during a pandemic.

18. The tangible machine-readable storage medium as recited in claim 17, wherein the predictive function is for a reproductive number R(t) during the pandemic, wherein R(t) is a linear function with respect to t.

19. The tangible machine-readable storage medium as recited in claim 17, Wherein a window of time is predefined for drawing samples of a number of cases in the pandemic.

20. The tangible machine-readable storage medium as recited in claim 16, wherein each first try t operation comprises:
drawing a test sample y_{t,c} from a proposal function Q)y_{t,c}|θ_{i,0}, . . . , θ_{i,N}_{_{c}}-1); and
calculating the likelihood function l_{y,t,c} for the drawn test sample y_{t,c}.
